(19) United States
(12) Patent Application Publication
(10) Pub. No.: US 2008/0206860 A1
(43) Pub. Date: Aug. 28, 2008

(54) Vessel and Culture System Including

(75) Inventors: William Armstrong, Hull (GB); Jean Armstrong, Hull (GB)

Correspondence Address: PATENT ADMINISTRATOR;KATTEN MUCHIN ROSENMAN LLP, 1025 THOMAS JEFFERSON STREET, N.W., EAST LOBBY: SUITE 700, WASHINGTON, DC, 20007-5201, US

(73) Assignee: THE UNIVERSITY OF HULL, Hull (GB)

(21) Appl. No.: 10/567,338

(22) PCT Filed: Aug. 3, 2004
(86) PCT No.: PCT/GB2004/003352

§ 371 Date: Apr. 11, 2008

(30) Foreign Application Priority Data
Aug. 6, 2003 (GB) 0318411.6

(51) Int. Cl. C12N 5/06; B01J 19/00; C12N 1/20; C12N 5/04; C12N 1/00; C12M 1/00

(52) U.S. Cl. 435/325; 422/255; 435/297.1; 435/252.1; 435/255.1; 435/410

(57) ABSTRACT

A vessel includes a gas reservoir. The vessel also includes at least one gas outlet. The gas outlet includes an integral gas permeable membrane. A culture system includes the vessel as a first vessel. The first vessel is coupled to a second vessel that includes or otherwise contains a cell, a plant, an animal or the like.

### BACKGROUND TO THE INVENTION

This invention relates to a vessel comprising a gas reservoir and at least one gas outlet, wherein said gas outlet comprises an integral gas permeable membrane and a culture system comprising said vessel.

Micro-propagation encompasses a range of tissue culture methods for the propagation of plant species. In essence, tissue from a plant (explant) is isolated to create a sterile culture of that species in vitro. Once a culture is stabilized and growing well multiplication of the tissue or regeneration of entire plants can be carried out. Shoots (tips, nodes or internodes) and leaf pieces are commonly used but cultures can be generated from many different tissues. This method of cultivation of plant material is generally used for rapid, large-scale, year round production of desired horticultural varieties; propagation of plant species that are difficult to grow from seed; production of genetically uniform plant material (“clones”); development of plant culture systems that can be used for genetic transformation, e.g. to introduce disease resistance and production of disease-free plant material.

Culture vessels currently employed for plant micropropagation allow only poor ventilation and inadequate supplies of carbon dioxide to the cultures, so that at best the plants only photosynthesise at very low rates (Argita et al., 2002; Buddendorf-Joosten et al., 1994; Kozai et al., 1991; Kozai et al., 1995 and Kozai et al., 1989). Poor ventilation can lead also to accumulations of the gaseous hormone ethylene which may cause vitrification and other abnormalities in sensitive species (George 1995).

Whilst some photosynthesis may be supported by CO₂ diffusion under the lid of the culture vessel from the growth room atmosphere, unless the plants are maintained under continuous illumination, net daily photosynthesis may not be possible. This necessitates the addition of sugar to the nutrient medium which sometimes induces plant abnormalities and encourages the growth of contaminants, leading to considerable plant losses and increased costs of producing plants (George, 1995). Furthermore, plants deprived of adequate levels of CO₂ may develop characteristics which lead to heavy losses at weaning to the glasshouse. These symptoms include (a) dysfunctional, gaping, stomata which tend to cause excessive water loss from the plant, (b) insufficient food reserves (e.g. starch) and (c) poor lignification (woodiness). One of the major aims of the micro-propagation industry worldwide is to promote the growth of fully photosynthesising plants in the laboratory, and so reduce or eliminate the need for additions of sugar to the nutrient medium (Figuiern and Janick, 1994; George, 1995; Kozai et al., 1991 and 1995).

GB 2,275,052 attempted to overcome the problem of poor ventilation in micro-propagation vessels by providing a ventilation apparatus and system for ventilating plant tissue cultures. The apparatus comprises a chamber having a wall made from microporous membrane and a means for maintaining a water vapour partial pressure inside the chamber exceeding that outside the chamber. This partial pressure differential induces a diffusive flow of atmospheric gases across the microporous membrane generating a positive pressure inside the chamber. An outlet discharges a continuous flow of humidified air from the chamber into a culture vessel. However whilst this system reduces ethylene accumulation, the rate of flow is often insufficient to maintain a high enough concentration of CO₂ in the culture vessels to keep pace with the scavenging demands of the plants. The situation can be partly remedied by increasing the flow potential of GB 2,275,052B but unrealistically high rates of flow may be needed even to raise culture vessel CO₂ concentrations to atmospheric levels. An alternative and more practical method is to enrich the ventilating stream itself with CO₂.

Current methods of delivering CO₂ to cultures in plant micro-propagation rely on complex systems involving gas cylinders, pumps, regulators, gas mixers and filters (Buddendorf-Joosten et al., 1994). A need therefore exists for a simple, inexpensive, portable method of delivering a sterile enriched gas to a culture vessel.

### STATEMENT OF THE INVENTION

Thus, according to a first aspect of the invention there is provided a vessel comprising a gas reservoir and at least one gas outlet, wherein said gas outlet comprises an integral gas permeable membrane.

Preferably the gas diffuses across the gas permeable membrane. This differs from the conventional gas cylinders, for example CO₂, in which the gas is transported by convection and a needle valve is required to control the bulk flow rate from the cylinder. The rate of diffusion across the gas permeable membrane is dependent on the membrane's diffusive resistance which can be varied by altering the physical properties of the membrane. Such alterations can be achieved by altering the surface area or nature of the membrane and/or by using a membrane of different wall thickness. The membrane may consist of silicon rubber (Si-rubber, which) has a CO₂ permeability coefficient, Pₛᵢ, of ca. 2.28×10⁻5 cm² s⁻¹@20° C. or other materials with a suitable diffusive resistance. The effective diffusive resistance, R_{D}, of an annulus of membrane length L, is given by: RD=(rᵢ logₑ rₒ/rᵢ)/Pₛᵢ2πiL, where rᵢ and rₒ are the inner radii of the Si-rubber tube. Similarly, the effective diffusive resistance for other gases can be determined by using their respective permeability coefficients in the above equation.

In a preferred embodiment of the invention the gas is a fluid, even more preferably the fluid is a gas, which is slected from the group consisting of, for example, O₂ or CO₂. In an alternative embodiment of the invention the fluid is a liquid. Even more preferably the gas reservoir is enriched for at least one gaseous species. Preferably still the gas reservoir is a liquid enriched for CO₂, for example, carbonated water or a solution of buffered bicarbonate salt which enriches for CO₂. During culture the vessel is linked to at least one substantially sealed culture vessel. The concentration of CO₂ in the gas reservoir diminishes during the culture period, as a result of use by the culture and leakage from the culture vessel. This diminishment is particularly advantageous during the micro-propagation of plants as it essentially ‘weans’ the plants off high concentrations of CO₂ so that at the completion of the micro-propagation phase they are able to readily adapt to atmospheric CO₂ levels.

If, however the rate of diffusion of CO₂ across the gas permeable membrane is to be maintained or increased during culture this may be achieved by (i) the use of more than one gas permeable membrane per vessel, (ii) the replacement of a membrane with a high diffusive resistance to one with a lower diffusive resistance or (iii) the use of a membrane with the potential to alter its diffusive resistance in response to conditions in the culture vessel.

In a further embodiment of the invention, the gas is sterilised as it diffuses across the gas permeable membrane.

In a still further embodiment of the invention the gas reservoir comprises more than one gaseous species. For example the gas reservoir may comprise CO₂ and a gaseous ethylene inhibitor. Ethylene is a very potent gaseous hormone produced by plants and can at times be toxic in plant micro-propagation cultures. It is frequently cited as a cause of abnormal growth or vitrification, including stunting, leaf curling and premature shoot senescence in sensitive species (George, 1995 and Righetti et al., 1990). The gaseous ethylene inhibitor 1-methyl cyclopropene (1-MCP) available from Rohm & Haas Co. (PA, USA), is an extremely specific inhibitor of ethylene action and works by binding specifically to the sites of ethylene action within the plant. By providing a 1-MCP solution in the gas reservoir the present invention can be used to enrich the culture vessel with gaseous 1-MCP and thus prevent vitrification.

According to a further aspect of the present invention there is provided a culture system comprising a vessel according to the invention, wherein said vessel is connected to a second vessel comprising a cell.

Preferably this is a plant cell. Even more preferably the plant is undergoing micro-propagation. Alternatively, the cell is selected from an animal, bacterial or yeast cell.

In a further aspect of the invention, there is provided a culture system comprising a first vessel according to the invention which is connected to a second vessel which contains a plant.

The vessel of the present invention may also be utilized for medical applications, specifically veterinary applications, wherein it may be used as part of a ventilation device/system to deliver and/or enrich oxygen to small animals/insects located within a second vessel.

Thus according to a still further aspect of the invention, there is provided a ventilation system comprising a first vessel according to the invention which is connected to a second vessel which contains an animal.

The vessel according to the invention can be connected by way of interconnecting means to more than one culture vessel.

Following diffusion through the gas permeable membrane the gas is transported towards the culture vessel by diffusion. This is suitable for plants which are not ethylene sensitive and therefore do not require a ventilation stream through the culture vessel. Alternatively, for plants that are ethylene sensitive and where a gaseous ethylene inhibitor is not being used, flushing out of potentially toxic gaseous products from the culture vessel can be achieved by further connecting a means of convective, pressurised delivery to the culture system. Thus in a further embodiment of the invention the culture system is further adapted to connect with a pressurised ventilation stream. For example, an interconnection between the first and second vessel, preferably in the form of a pipeline, may be adapted to connect with the pressurised ventilation stream. The connection may be to the outflow tube of a pressure-flow source such as a humidity-induced forced ventilation apparatus described in GB 2,275,052 or some other filtered air source.

In a culture system reliant on the simple diffusive delivery of the gas the rate of flow of the gas from the gas reservoir to the culture vessel is dependent on the following factors; (i) the diffusive resistance of the gas-permeable membrane, (ii) the diffusive resistance of the connection, as well as the diffusive resistance under the rim.

In a culture system reliant on convective, pressurised delivery the rate of flow from the gas reservoir to the culture vessel depends chiefly on (i) the diffusive resistance of the gas-permeable membrane and (ii) the rate of convective gas flow from the pressurised ventilating source.

According to a further aspect of invention a method is provided for the supply of a gaseous species to a cell comprising the steps of;
(i) providing a vessel comprising a gas reservoir and at least one gas outlet wherein the gas outlet comprises a gas-permeable membrane;
(ii) connecting, via an interconnecting means, the vessel to at least a second vessel comprising a cell; and optionally,
(iii) further connecting a humidity-induced forced ventilation apparatus to said interconnecting means.

Preferably the method is used in the supply of a gaseous species to a cell. Preferably this cell is a plant cell. en more preferably this plant cell is undergoing micro-propagation. In an alternative preferred method, a gaseous species is supplied to a cell selected from the group consisting of an animal, bacterial or yeast cell.

### BRIEF DESCRIPTION OF THE DRAWINGS

The invention will be more clearly understood from the following description of some embodiments thereof, given by way of example only, with reference to the accompanying drawings, in which:

FIG. 1: Schematic of the culture apparatus for delivery of gas from a vessel comprising a gas reservoir to a culture vessel by simple diffusive flow (Method A).

FIG. 2: Schematic of the culture apparatus for delivery of gas from a vessel comprising a gas reservoir to a culture vessel by convective, pressurised flow (Method B).

FIG. 3: Illustrates an example of gas delivery by convective, pressurised flow [Method (B)] when there is not a sample in the culture vessel.

FIG. 4: These graphs which are derived from mathematical modelling, illustrate how the diffusive resistance of the gas permeable membrane (as a function of its length) affects photosynthesis of micro-propagated Cherry when applying CO₂-enrichment by Method (A) and Method (B).

FIG. 5: Illustrates details of CO₂ supply rate and escape rate of unused CO₂ from the culture vessels outlined in FIG. 4.

FIG. 6: Illustrates daily net photosynthesis of Cherry grown on multiplication media either with (a) forced ventilation in conjunction with CO₂ enrichment from the culture apparatus of the present invention, (b) forced ventilation without CO₂ enrichment or (c) conventional diffusive ventilation.

Table 1: Illustrates the beneficial effects obtained by applying the gaseous ethylene inhibitor; 1-methyl cyclopropene (1-MCP) to cherry using the culture apparatus of the present invention.

### DETAILED DESCRIPTION OF THE DRAWINGS

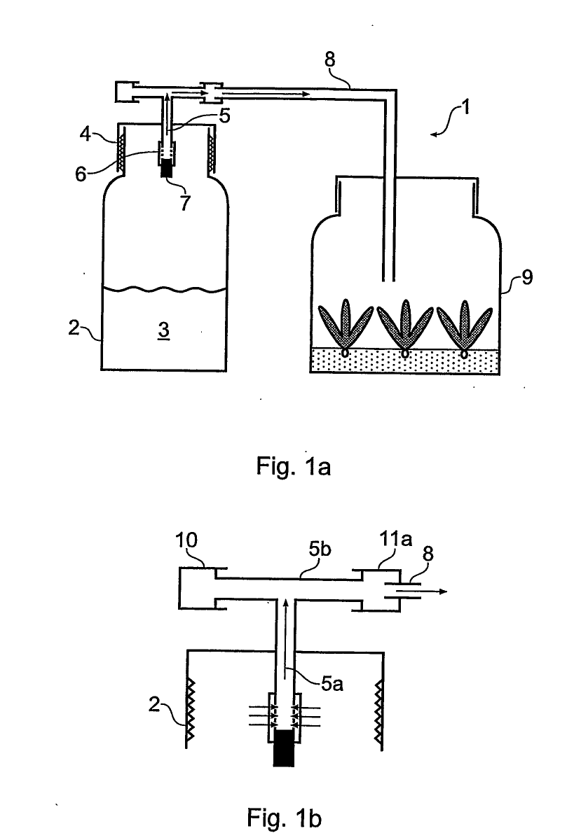

FIG. 1.

(a) This schematic illustrates the culture system 1 when used for simple, diffusive delivery of a gas. The vessel 2 comprises a gas reservoir 3 sealed from the atmosphere by the vessel having a gas-tight, screw-top lid 4. This gas reservoir 3 may, for example, be carbonated water or a solution of buffered bicarbonate salt (e.g sodium bicarbonate). A gas outlet 5 with an integral gas permeable membrane 6 is positioned in the head space of the vessel 2. The lower end of this gas outlet is sealed with a blanking plug of glass or other material 7. As the gas diffuses across the membrane and out of the vessel it enters an interconnecting means 8 and diffuses in the direction of the arrows towards a culture vessel 9.

(b) This schematic illustrates a magnified view of the gas outlet S extending from the vessel 2 comprising the gas reservoir. In this embodiment the gas outlet is a T-piece with a vertical limb 5a and a horizontal limb 5b. When in use the end of limb 5b which is distal to the culture vessel is sealed by means of a blanking screw 10, whilst that which is promixal is attached by a screw connector 11a to the interconnecting means 8 (e.g a plastic tube), of low wall permeability to gases, which extends through the lid of the culture vessel.

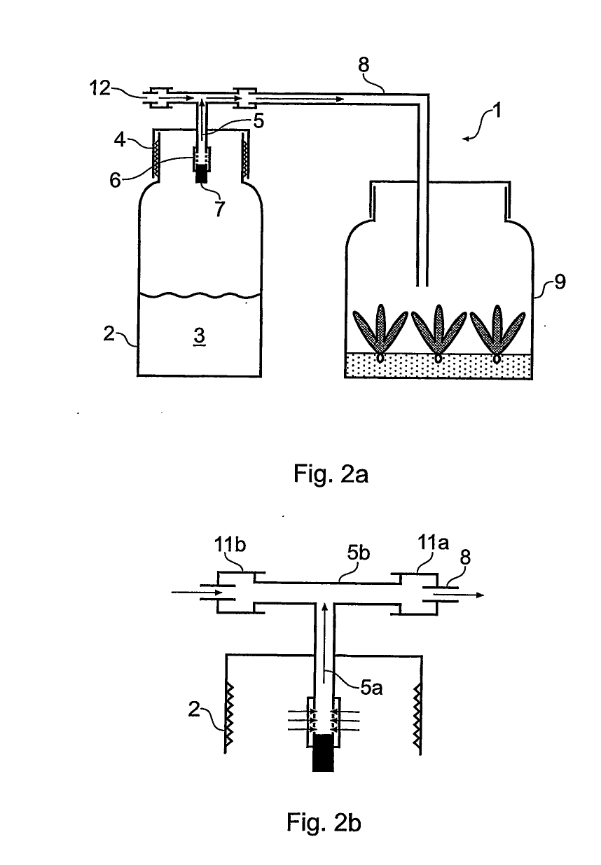

FIG. 2.

(a) This schematic illustrates the culture system 1 when using convective, pressurised gas delivery. As the gas diffuses through the gas outlet 5 it mixes with the outflow from a pressure-flow source 12, such as a humidity-induced forced ventilation apparatus. The arrows indicate the direction of convective gas flow along the interconnecting means 8.

(b) This schematic illustrates a further embodiment of the invention and shows a magnified view of the gas inlet 5 extending from the vessel comprising the gas reservoir. In this embodiment the gas outlet is a T-piece with a vertical limb 5a and a horizontal limb 5b. When in use the end of limb 5b which is distal to the culture vessel is attached by means of a screw connector 11b to the outflow of a pressure-flow source 12 such as a humidity-induced forced ventilation apparatus, The end which is promixal is attached by a screw connector 11a to the interconnecting means 8 (e.g a plastic tube), of low wall permeability to gases, which extends through the lid of the culture vessel.

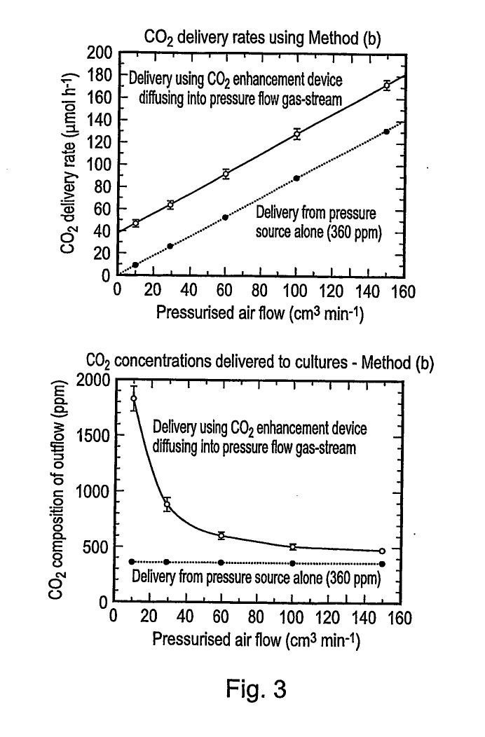

FIG. 3: Illustrates an example of gas delivery by convective, pressurised flow [Method (B)] when there is not a sample in the culture vessel. It illustrates the effects of increasing the rate of pressurised air flow through the T-piece on both CO₂ delivery rate and CO₂ concentration. A comparison is made with apparatus without a CO₂-enrichment device.

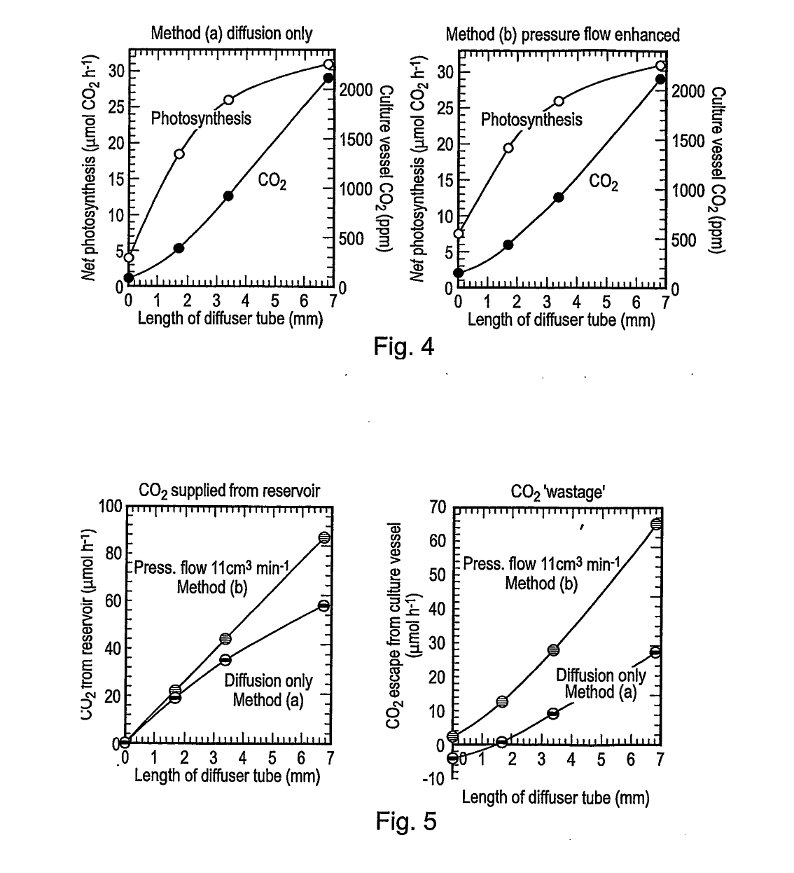

FIG. 4: These graphs which are derived from mathematical modelling, illustrate how the diffusive resistance of the gas permeable membrane (as a function of its length) affects photosynthesis of micro-propagated Cherry when applying CO₂-enrichment by Method (A) and Method (B). It can be seen that by both procedures, photosynthesis is much enhanced above that obtained without CO₂-enrichment. With a gas permeable membrane length of 3 mm, photosynthesis is 6.5 times greater than could be achieved by conventional treatments that rely on CO₂ diffusion from the growth room atmosphere under the lid of the culture vessel. However, with Method (a) more of the CO₂ used by the plants is derived from the reservoir than with Method (b) where the pressurised gas flow itself can contain some CO₂; in the examples shown the pressurised gas stream contained atmospheric levels (360 ppm). As the gas permeable membrane length is increased (which lowers its diffusive resistance, Method (b) removes increasingly more CO₂ from the reservoir than Method (a) and proportionally less of it is used in photosynthesis. Overall Method (b) is more wasteful of CO₂ than Method (a) but this is unavoidable if a pressure flow gas stream is necessary also to remove undesirable volatiles from the culture vessels.

FIG. 5: Illustrates details of CO₂ supply rate and escape rate of unused CO₂ from the culture vessels outlined in FIG. 4. Overall Method (b) is more wasteful of CO₂ than Method (a) but this is unavoidable if a pressure flow gas stream is necessary also to remove undesirable volatiles from the culture vessels.

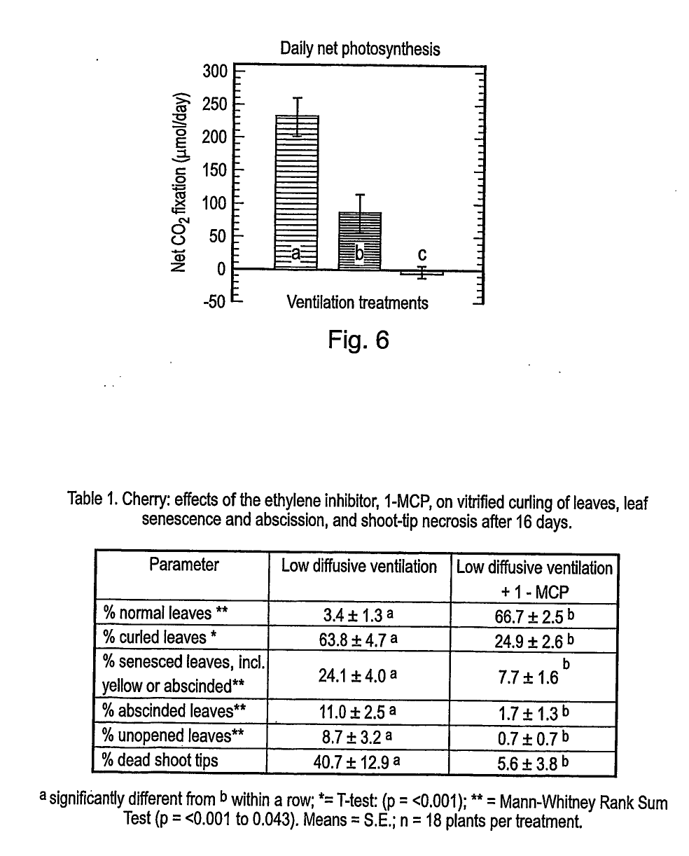

FIG. 6: Illustrates daily net photosynthesis of Cherry grown on multiplication media either with (a) forced ventilation in conjunction with CO₂ enrichment from the culture apparatus of the present invention, (b) forced ventilation without CO₂ enrichment or (c) conventional diffusive ventilation. When used for CO₂ enrichment, this culture system stimulates photosynthesis.

Table 1:

Illustrates the effect of the ethylene inhibitor, 1-MCP on vitrified curling of leaves, leaf senescence and abscission and shoot tip necrosis after 16 days.

### REFERENCES

(1) Arigita L., González A., Tamés R. S. (2002). Influence of CO₂ and sucrose on photosynthesis and transpiration of Actinidia deliciosa explants cultured in vitro. Physiologia Plantarum 115, 166-173.

(2) Buddendorf-Joosten, J. M. C., Woltering, E. J. 1994. Components of the gaseous environment and their effects on plant growth and dvelopment in vitro. Plant Growth Regulation 15, 1-16.

(3) Figueira A, Janick J. 1994. Optimizing carbon dioxide and light levels during in vitro culture of Theobroma cacao. J. Amer. Soc. Hort. Sci. 119, 865-871.

(4) George, E. F. (1995). Plant Propagation by Tissue Culture. Exogenetics Ltd., Edington, England.

(5) Kozai T., Iwabuchi K, Watanabe K, Watanabe I. (1991). Photoautotrophic and photomixotrophic growth of strawberry plantlets in vitro and changes in nutrient composition of the medium. Plant Cell Tissue & Organ Culture 25, 107-115.

(6) Kozai, T., Kitaya, Y. and Kubota, C. (1995). Collected papers on Environmental Control in Micropropagation, Vol. 3 (1994-1995). Genhua Niu, ed. Laboratory of Environmental Control Engineering, Faculty of Horticulture, Chiba University, Chiba 271, Japan. 404 pages.

(7) Kozai, T., Kubota, C. and Nakayama, M. (1989). Net photosynthetic rates of plantlets in vitro under natural and forced ventilation conditions. Annual Meeting, Japanese Society of Horticultural Science. pp. 250-251

(8) Righetti, B., Magnanini, E., Infante, R. and Predieri, S. (1990). Ethylene, ethanol, acetaldehyde and carbon dioxide released by Prunus avium shoot cultures. Physiologia Plantarum. 78:507-510.

(9) University of Hull with Armstrong, J. and Armstrong, W. (1996). Ventilation Apparatus and System—being an apparatus for the humidified ventilation of plant tissue culture or of animal bums using Nuclepore membranes to obtain a humidity-induced diffision and consequent convection. U.K. Patent No. GB-2275052B.

1. A vessel comprising a gas reservoir and at least one gas outlet, wherein said gas outlet comprises an integral gas permeable membrane.

2. A vessel according claim 1, wherein the gas flow across the gas permeable membrane is by diffusion.

3. A vessel according to claims 1 or 2, wherein diffusion of the gas through the gas permeable membrane sterilises the gas.

4. A vessel according to any of claims 1 to 3, wherein the gas reservoir is a fluid.

5. A vessel according to claim 4, wherein the fluid is a gas.

6. A vessel according to claim 5, wherein the gas is selected from either O₂ or CO₂.

7. A vessel according to claim 4, wherein the fluid is a liquid.

8. A vessel according to claim 7, wherein the liquid is gas enriched.

9. A vessel according to claims 7 or 8, wherein the liquid is CO₂ enriched.

10. A vessel according to claim 9, wherein the CO₂ enriched liquid is selected from either carbonated water or a solution of buffered bicarbonate salt.

11. A vessel according to any of claims 1-10, wherein the gas reservoir comprises more than one gaseous species.

12. A vessel according to any of claims 1-11, wherein the gaseous reservoir further comprises an ethylene inhibitor.

13. A vessel according to claim 12, wherein the ethylene inhibitor is 1-methyl cyclopropene.

14. A culture system comprising a first vessel according to any of claims 1 to 13, wherein the first vessel is connected to a second vessel which comprises a cell.

15. A culture system according to claim 14, wherein the cell is a plant cell.

16. A culture system according to claims 14 or 15, wherein the plant cell is undergoing micro-propagation.

17. A culture system according to claim 14, wherein the cell is an animal cell.

18. A culture system according to claim 14, wherein the cell is a bacterial cell.

19. A culture system according to claim 14, wherein the cell is a yeast cell.

20. A culture system comprising a first vessel according to any of claims 1-13, wherein the first vessel is connected to a second vessel which contains a plant.

21. A ventilation system comprising a first vessel according to any of claims 1-11, wherein the first vessel is connected to a second vessel which contains an animal.

22. A culture system or ventilation system according to any of claims 14-21, wherein the gas outlet on the first vessel is adapted to connect with a pressurised ventilation stream.

23. A culture system or ventilation system according to claim 22, wherein the pressurised ventilation stream is derived from a humidity-induced forced ventilation apparatus.

24. A method for the supply of a gaseous species to a cell, comprising the steps of;
i) providing a vessel comprising a gas reservoir and at least one gas outlet wherein the gas outlet comprises a gas-permeable membrane;
ii) connecting, via an interconnecting means, the vessel to at least a second vessel comprising a cell; and optionally,
iii) further connecting a humidity-induced forced ventilation apparatus to said interconnecting means.

25. A method according to claim 24, wherein the cell is a plant cell.

26. A method according to claim 25, wherein the plant cell is undergoing micro-propagation.

27. A method according to claim 24, wherein the cell is selected from the group consisting of an animal, bacterial or yeast cell.

28. A vessel, culture system or a method substantially as described with reference to the accompanying examples.
